(19) United States
(12) Patent Application Publication
(10) Pub. No.: US 2009/0259127 A1
(43) Pub. Date: Oct. 15, 2009

(54) Disposable High Frequency Multi-Focus Silicone Self-Contained Water Bath Offset

(75) Inventor: Timothy J. Ehrecke, Bettendorf, IA (US)

Correspondence Address: HODGSON RUSS LLP;THE GUARANTY BUILDING, 140 PEARL STREET, SUITE 100, BUFFALO, NY, 14202-4040, US

(73) Assignee: REICHERT, INC., Depew, NY (US)

(21) Appl. No.: 12/402,798

(22) Filed: Mar. 12, 2009

Related U.S. Application Data
(60) Provisional application No. 61/035,787, filed on Mar. 12, 2008.

(51) Int. Cl. A61B 8/14

(52) U.S. Cl. 600/459

(57) ABSTRACT

A multiple focal length transducer comprising a first end allowing cooperable attachment to an ultrasound bio-microscope. A second end allowing contact with a water bath. An adjustable connecting portion allowing multiple focal lengths. The adjustable connecting portion may be allowed to flex similar to an accordion. The multiple focal length transducer is preferably produced to be sterile from medical grade silicone and is disposable.

### CROSS REFERENCE TO RELATED APPLICATIONS

None

### FIELD OF INVENTION

An ultrasonic transparent transducer useful in the fields of Opthalmology and Optometry is disclosed and claimed herein. The transducer is particular useful in combination with a biomicroscope.

### STATEMENT REGARDING FEDERALLY SPONSORED RESEARCH OR DEVELOPMENT

No federal funds were used to develop or create the invention disclosed and described in the patent application.

### REFERENCE TO SEQUENCE LISTING, A TABLE, OR A COMPUTER PROGRAM LISTING COMPACT DISK APPENDIX

Not Applicable

### AUTHORIZATION PURSUANT TO 37 C.F.R. §1.171 (d)(c)

A portion of the disclosure of this patent document contains material which is subject to copyright and trademark protection. The copyright owner has no objection to the facsimile reproduction by anyone of the patent document or the patent disclosure, as it appears in the Patent and Trademark Office patent file or records, but otherwise reserves all copyrights whatsoever.

### BACKGROUND OF INVENTION

An ultrasound bio-microscope (UBM) is used when assessing a patient's conditions before surgery, during an exam or post surgery. For example, when the cornea of an eye is opaque, because of disease or damage, it is not possible for surgeons to know what they're going to find inside the eye during a procedure, for example. Images of the eye are produced using the UMB, particularly high resolution images of the anterior chamber of the eye. Typically, the UMB operates at 30-50 MHz. This is useful in finding and or interpreting whether a disease or condition exists. For example, an iris cyst or tumor may be detected using a UBM.

An ultrasound bio-microscope, works by covering the eye with a thin layer of water; a transducer probe then oscillates in the water, using ultrasound technology to produce an image of the inside of the eye. During the procedure, the patient must lay down in order to facilitate creation of the water bath. At present in the prior art, to vary the focal length of the transducer, the transducer must be removed and a water bath again created around the patient's eye to facilitate use of the ultrasound bio-microscope. Because of the position of the patient and the need to create a water bath upon and around the patient's eye, a transducer allowing multiple focal lengths with minimal disruption to the water bath would be beneficial and desirable.

### SUMMARY OF INVENTION

A disposable high frequency multi-focus silicone water bath offset (transducer) is claimed and disclosed herein. The transducer may be used in a self-contained water bath offset. The transducer has a baffled or accordion type wall structure that allows it be adjusted for multiple focal lengths. The transducer is preferably sterile and produced from soft, resilient medical grade silicone. Other materials that have similar hardness properties and germ resistant may also be selected, dependent on the particular application. To enhance the germ resistance and to maintain sterility, the transducer may also be produced as a disposable unit.

### BRIEF DESCRIPTION OF THE FIGURES

FIG. 1 provides an illustration of the how an ultrasound bio-microscope of the prior art works with a fixed wall transducer of the prior art.

FIG. 2 illustrates the present art disposable high frequency multi-focus silicone water bath offset (transducer) claimed and disclosed herein.

FIG. 3 provides a photo of an UBM known in the art and produced by Applicant.

Before the various embodiments of the present invention are explained in detail, it is to be understood that the invention is not limited in its application to the details of construction and the arrangements of components set forth in the following description or illustrated in the drawings. The invention is capable of other embodiments and of being practiced or of being carried out in various ways. Also, it is to be understood that phraseology and terminology used herein with reference to device or element orientation (such as, for example, terms like “front”, “back”, “up”, “down”, “top”, “bottom”, and the like) are only used to simplify description of the present invention, and do not alone indicate or imply that the device or element referred to must have a particular orientation. In addition, terms such as “first”, “second”, and “third” are used herein and in the appended claims for purposes of description and are not intended to indicate or imply relative importance or significance.

### DETAILED DESCRIPTION

### Listing of Elements

| ELEMENT DESCRIPTION | ELEMENT # |
|---|---|
| First End | 1 |
| Second End | 2 |
| Ring | 3 |
| Wall | 4 |
| Multiple Focal Length Transducer | 5 |
| Intentionally Blank | 6 |
| Intentionally Blank | 7 |
| Intentionally Blank | 8 |
| Ultrasound Biomicroscope (UBM) | 9 |
| Tool | 10 |
| Water bath | 11 |
| Adjustable Connecting Portion | 12 |
| Focal Length | 13 |

### DETAILED DESCRIPTION

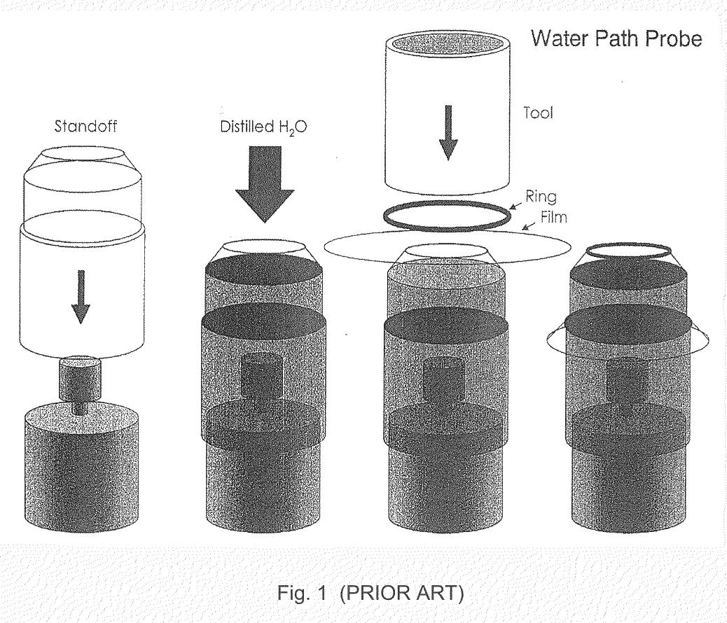

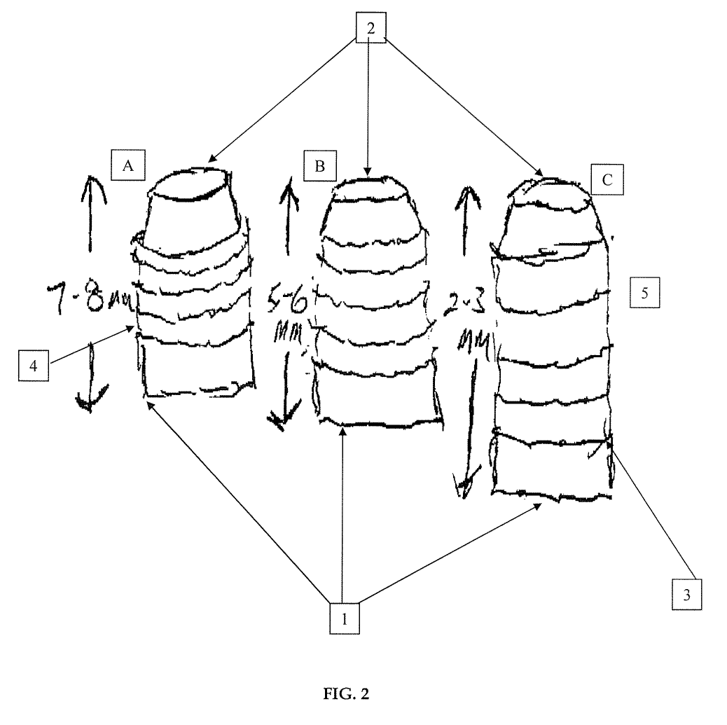

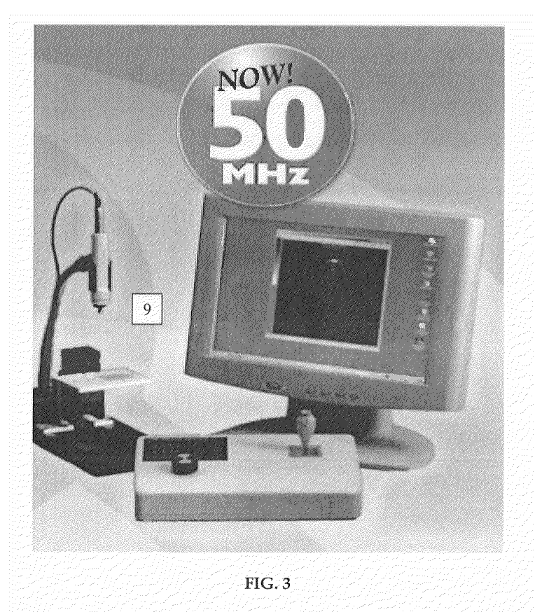

Referring now to the drawings, wherein like reference numerals designate identical or corresponding parts throughout the several views, FIG. 1 provides an illustration of the prior art transducer working with an ultrasound bio-microscope (UBM). FIG. 3 provides a photo of an UBM known in the art. Transducers of the prior art have fixed walls. To change the focal length of the ultrasound biomicroscope, the transducer may be removed from the bath and replaced with one having a different thickness and or focal length. FIG. 2 illustrates the present art disposable high frequency multi-focus silicone water bath offset (transducer) claimed and disclosed herein. The multiple focus transducer 5 comprises a generally cylindrical structure having a first end 1 and a second end 2. The first end may be engaged with a tool 10 connected to and in communication with an UBM 9. The second end 2 may be engaged with an eye. At least one ring 3 is positioned between the first and second ends (1, 2). The at least one ring 3 allowing compression or decompression which in turn changes the focal length 13 of the multiple focus transducer 5. By way of illustration, and without limitation, the multiple focal length transducer 5, shown at A, has a focal length of 7-8 mm. By decompressing or elongating the multiple focal length transducer 5, shown at B, the focal length of 7-8 millimeters has been decreased to 5-6 millimeters. By further decompressing or elongating the multiple focal length transducer 5, shown at C, the focal length of 5-6 millimeters has been further decreased to 2-3 millimeters. The embodiment illustrated at FIG. 2 has an outer wall that is composed of rings that allow the elongation or compression of the transducer body to affect changes to the focal length. Although not shown, other methods and structures that allow for a multiple focal length transducer 5 with an adjustable connecting portion 12 between the first and second end which allows for multiple focal lengths because of the accordion like flexing of the flexible outer walls of the transducer. Although not shown, the outer walls of the transducers of the prior art are typically rigid. The multiple focus transducer 5 is preferably produced from medical grade silicone, is either sterile, disposable or both.

FIG. 3 illustrates a UMB that may be used with the present art. As shown, the UBM allows for high frequency ultrasound ophthalmic diagnostics. A high resolution image is produced through non-invasive means. Some applications for technology of this type include, by way of illustration and without limitation:
Developmental Biology
Genomic Research
Cardiovascular Research
Dermatology
Veterinary Ophthalmic Diagnostics

As shown the UBM may be used with the present for direct contact through the probe attachment and a traditional water bath.

It should be noted that the present invention is not limited to the specific embodiments pictured and described herein, but is intended to apply to all similar apparatuses for imaging an eye using ultrasound or ultrasonic imaging technologies. Modifications and alterations from the described embodiments will occur to those skilled in the art without departure from the spirit and scope of the present invention.

1. A multiple focal length transducer 5 comprising:
a. A first end 1, said first end 1 allowing cooperable attachment to an ultrasound bio-microscope 9;
b. A second end 2, said second end 2 allowing contact with a water bath 11;
c. An adjustable connecting portion 12, said adjustable connecting portion 12 allowing multiple focal lengths.

2. The multiple focal length transducer 5 according to claim 1 wherein said adjustable connecting portion 12 allows accordion like flexing.

3. The multiple focal length transducer 5 according to claim 1 wherein said multiple focal length transducer 5 is produced from medical grade silicone.

4. The multiple focus transducer 5 of claim 1 wherein said multiple focal length transducer 5 is disposable.

5. The multiple focus transducer 5 of claim 1 wherein said multiple focal length transducer 5 is sterile.

6. The multiple focus transducer 5 of claim 4 wherein said multiple focal length transducer 5 is sterile.

7. A multiple focus transducer 5 comprising:
a. A generally cylindrical structure having a first end 1 and a second end 2, wherein said first end may be engaged with a tool 10 connected to and in communication with an ultrasound bio-microscope 9, wherein said second end 2 may be engaged with an eye;
b. At least one ring 3, said at least one ring 3 positioned between said first and said second ends (1, 2), said at least one ring 3 allowing compression or decompression, and wherein said compression or decompression of said at least one ring changes the focal length 13 of said multiple focus transducer 5.

8. The multiple focus transducer 5 of claim 7 wherein said multiple focus transducer 5 is produced from medical grade silicone.

9. The multiple focus transducer 5 of claim 7 wherein said multiple focus transducer is disposable.

10. The multiple focus transducer 5 of claim 7 wherein said multiple focus transducer is sterile.

11. The multiple focus transducer 5 of claim 9 wherein said multiple focus transducer is sterile.

12. A method of comprising any new, inventive step, act, combination of steps and or acts or sub-combination of steps and/or acts described herein related to multiple focal length transducers.

13. An apparatus comprising any new, inventive step, act, combination of steps and or acts or sub-combination of steps and/or acts described herein related to multiple focal length transducers.
